(19) United States
(12) Patent Application Publication
(10) Pub. No.: US 2024/0328945 A1
(43) Pub. Date: Oct. 3, 2024

(54) DETECTION METHOD FOR PROTEIN LIQUID-LIQUID PHASE SEPARATION AND USE THEREOF

(71) Applicant: SHENZHEN INSTITUTES OF ADVANCED TECHNOLOGY, Shenzhen (CN)

(72) Inventors: Leo E. WONG, Shenzhen (CN); Shuwen CHANG, Shenzhen (CN)

(73) Assignee: SHENZHEN INSTITUTES OF ADVANCED TECHNOLOGY, Shenzhen (CN)

(21) Appl. No.: 18/741,387

(22) Filed: Jun. 12, 2024

Related U.S. Application Data
(63) Continuation of application No. PCT/CN2021/137736, filed on Dec. 14, 2021.

(51) Int. Cl. G01N 21/64; C12N 13/00

(52) U.S. Cl. CPC G01N 21/6428; C12N 13/00; G01N 2021/6439

(57) ABSTRACT

Disclosed are a detection method for protein liquid-liquid phase separation (LLPS) and use thereof. The method comprises: constructing a first expression vector for expressing an energy donor fluorescent molecule fused to a target protein and a second expression vector for expressing an energy acceptor fluorescent molecule fused to the target protein; transferring both expression vectors into primary cultured cells; stimulating the primary cultured cells while collecting fluorescence microscopic imaging data by using an appropriate fluorescence microscope; calculating a change in Förster resonance energy transfer (FRET) efficiency as a proxy for monitoring the process of LLPS and phase transition of the target protein.

### TECHNICAL FIELD

The present invention relates to the field of biotechnologies, and in particular, relates to a detection method for protein liquid-liquid phase separation and use thereof.

### BACKGROUND

Research shows that many kinds of neuronal synaptic proteins can be liquid-phase separated in an in-vitro experiment, and some of them have a direct relationship with neurodegenerative diseases. Therefore, some researchers have proposed a theory that out-of-control protein phase transitions cause neurodegenerative diseases. However, this theory can only be proven by direct observation and characterization of liquid phase separation of a protein in a neuronal synapse, and on this ground it is possible to develop a new research direction.

Existing methods for detecting liquid phase separation of proteins in an in-vitro experiment include fluorescence microscopy and light scattering methods. Among them, the most common method is to observe the formation of submicron- to micron-sized objects in the form of droplets (i.e., condensed phases) using a fluorescence microscope by adjusting various physicochemical conditions (e.g., temperature, protein concentration, and salt concentration), and then measuring the mobility of the protein in the condensed phases using a fluorescence recovery after photobleaching (FRAP) technique. At the same time, the formation of the condensed phases also increases light scattering within the visible wavelength range, and thus the liquid phase separation of a protein can be detected using this method.

Neuronal synapses are the structural and functional units for the interconnection and communication between all neurons, with a diameter thereof being only 200-800 nanometers, and therefore, it is impossible to detect the liquid phase separation of proteins in synapses by observing the formation of submicron-to micron-sized condensed phases. Currently, some researchers have attempted to observe nanoscale clusters formed by proteins using various super-resolution fluorescence imaging techniques. However, super-resolution fluorescence imaging has high hardware requirements (microscopes are expensive) and application limitations (typically for immobilized cell samples). As both the photoactivated localization microscopy (PALM) and the stochastic optical reconstruction microscopy (STORM) are methods for acquiring localization based on repeatedly exciting, observing and bleaching a single activated fluorescent molecule plus calculation, their imaging speed has an upper limit, and the powerful laser used in stimulated emission depletion (STED) microscopy may cause damage to cells, so these methods are not beneficial to detecting the liquid phase separation process in living neurons.

### SUMMARY

The present invention aims to achieve the detection and tracking of the process of a protein undergoing LLPS or transition between different phase-separated states in a neuronal synapse on a sub-second time scale under the condition of receiving various forms of nerve stimulations, and provides a detection method in general for different phase-separated states of a protein and use thereof to solve the defects in the prior art. The process of a protein phase transition (i.e., from no liquid phase separation to different phase-separated states) may result in a change in the Förster resonance energy transfer efficiency between the proteins fused to energy donor fluorescent molecule and energy acceptor fluorescent molecule, respectively, and thus the protein phase transition can be detected by measuring and calculating the change in the energy transfer efficiency.

A first aspect of the present invention provides a detection method for protein liquid-liquid phase separation (LLPS) and phase transition which includes the following steps:
constructing a first expression vector for expressing an energy donor fluorescent molecule fused to a target protein and a second expression vector for expressing an energy acceptor fluorescent molecule fused to the target protein;
transferring the first and the second expression vector into primary cultured cells;
stimulating the primary cultured cells while collecting fluorescence imaging data by using an appropriate fluorescence microscope; and
calculating a change in Förster resonance energy transfer (FRET) efficiency as a proxy for monitoring the process of LLPS and phase transition of the target protein.

Further, the target protein is a neuronal synaptic protein;
the primary cultured cells are primary cultured neurons.

Further, the second expression vector comprises 2-3 energy acceptor fluorescent molecule nucleotide sequences in tandem.

Further, the primary cultured cells transfected with the first and the second expression vectors therein are stimulated by an electric field produced by an electric field generator;
preferably, the electric field has a strength of 25-50 V/cm and a frequency of 20-50 Hz, stimulating neural activity.

Further, the fluorescence microscope is installed with two or more excitation lasers or emission filters.

Further, the Förster resonance energy transfer efficiency is determined based on a sensitized emission method, a ratiometric method, or a fluorescence lifetime imaging (FLIM) method;
preferably, the Forster resonance energy transfer efficiency is determined based on the sensitized emission method.

Further, the detection method is used for detecting a process of LLPS and phase transition of a protein in the synapse of living neurons.

A second aspect of the present invention provides use of the detection method in drug target screening.

The present invention has the following beneficial effects:

1. The detection method for protein liquid phase separation is based on the principle of Förster resonance energy transfer, the efficiency of fluorescence resonance energy transfer is increased according to a shortened distance between molecules of a protein in condensed phases after protein liquid phase separation, the energy donor fluorescent molecule fused to the target protein as well as the energy acceptor fluorescent molecule fused to the target protein are expressed in the primary cultured cells, followed by various forms of stimulation, the fluorescence microscopic imaging data are then collected using a fluorescence microscope, the change in the Förster resonance energy transfer efficiency is calculated, and thus the process of protein liquid phase separation is detected. The detection method of the present invention achieves the detection of liquid phase separation of a protein in vitro or in living nerve cells (namely neurons), can be used for researching the effect of protein phase transitions on neurophysiology and pathology, and can be used for researching the phase transition process of a target protein on neurons of different genotypes or phenotypes, thereby discovering new disease mechanisms and drug targets.

In a preferred solution, by linking multiple energy acceptor fluorescent molecules in series, the efficiency of fluorescence resonance energy transfer is further increased, and the accuracy of detecting the liquid phase separation of the protein in the neuronal synapse is enhanced.

2. The present invention achieves the detection of liquid phase separation of a protein in a neuronal synapse on a sub-second time scale for the first time. The present invention detects a phase transition of the protein based on the fluorescence resonance energy transfer phenomenon caused by the shortened distance between protein molecules after the protein liquid phase separation, which is not limited by the size of the phase-separated condensates and can be used for detecting the liquid phase separation in a neuronal synapse with a diameter of only 200-800 nanometers. Secondly, the measurement of fluorescence resonance energy transfer can be technically completed on a sub-second time scale, which circumvents the imaging speed limit in PALM and STORM super-resolution fluorescence imaging technique and avoids the damage of powerful laser to cells in STED microscopy.

### BRIEF DESCRIPTION OF DRAWINGS

FIG. 1 shows the principle for detection of protein LLPS and phase transition as claimed in the present invention. In the figure, A. a target protein linked to an energy donor fluorescent molecule; B. the target protein linked to an energy acceptor fluorescent molecule; C. the target protein linked to multiple energy acceptor fluorescent molecules in series; D. a primary cultured neuron that has expressed proteins A and B (or A and C); E. various types of fluorescence microscopes; F. an electric field generator for stimulating the neuron; and G. neurons of different genotypes or phenotypes.

FIG. 2 shows experimental data demonstrating the principle and feasibility of the present invention in a COS-7 cell line. (Left graph) Fluorescence imaging shows that spherical condensed phases were formed after LLPS of the target protein; (right graph) pixel-wise calculated FRET efficiency based on the sensitized emission method shows a higher FRET efficiency (average 0.22) within the condensed phases as compared to the outside of the condensed phases (average 0.11).

### DETAILED DESCRIPTION OF THE EMBODIMENTS

In order to understand the present invention more clearly, the present invention will be further described with reference to the following examples and drawings. The examples are given for the purpose of illustration only and are not intended to limit the present invention in any way. In the examples, all of the reagents and starting materials are commercially available, and the experimental methods without specifying the specific conditions are conventional methods with conventional conditions well known in the art, or conditions suggested by the instrument manufacturer.

### Example 1

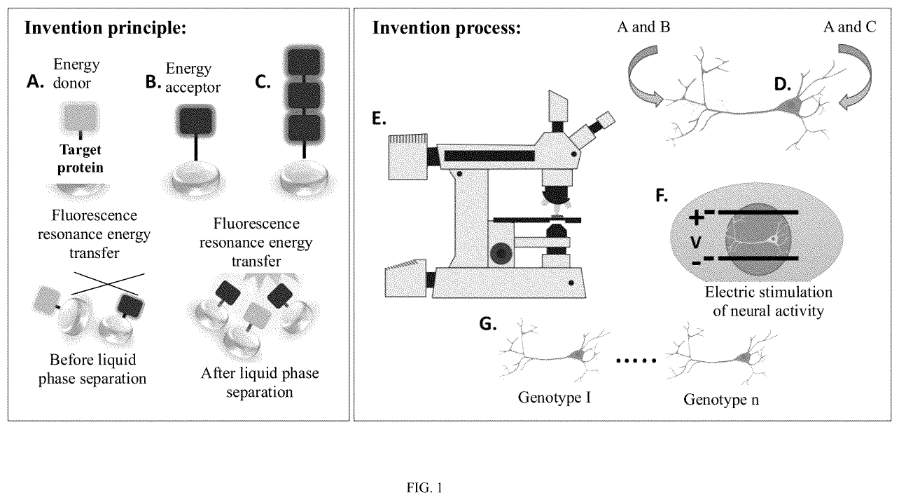

The present invention is based on the principle of Förster Resonance Energy Transfer (FRET), whereby an enhancement of FRET efficiency of a target protein can be detected during LLPS or phase transition. The detection principle and the flowchart of the procedures are shown in FIG. 1. A is a target protein linked to an energy donor fluorescent molecule, B is the target protein linked to an energy acceptor fluorescent molecule, and C is the target protein linked to multiple energy acceptor fluorescent molecules. D is a primary cultured neuron that has expressed proteins A and B (or A and C). E is a confocal laser scanning microscope (or other type of fluorescence microscope) with two or more excitation lasers and emission filters. F is an electric field generator capable of stimulating a neuron by producing an electric field. G is neurons of different genotypes or phenotypes.

The FRET efficiency depends on the distance between the energy donor (A) and the acceptor molecule (B or C). As liquid-liquid phase separation of a protein is driven by self-interaction between the molecules, it is predicted that the distance between the protein molecules in the condensed phases is shortened, thereby increasing the efficiency of FRET. The method can achieve the detection of LLPS or phase transition of a protein in neurons of different genotypes or phenotypes.

In a preferred solution, by linking multiple energy acceptor fluorescent molecules in series, the efficiency of FRET is further increased, and the detection sensitivity of the protein LLPS and phase transition in the neuronal synapse is thereby enhanced.

The detection method for protein LLPS and phase transition includes the following steps:

(1) a first expression vector for expressing an energy donor fluorescent molecule fused to a target protein and a second expression vector for expressing an energy acceptor fluorescent molecule fused to the target protein were constructed, and the first expression vector and the second expression vector were transfected into primary cultured neurons. In a preferred embodiment, the second expression vector comprises 1-3 energy acceptor fluorescent molecule nucleotide sequences in tandem.

(2) The neuron was stimulated by an electric field produced by an electric field generator.

(3) Fluorescence microscopic imaging data were collected using a confocal laser scanning microscope.

(4) The change in FRET efficiency was calculated based on a sensitized emission method, and thus the detection of the whole phase transition process of the target protein in the neuron was completed.

### Example 2

This example was validated in a COS-7 cell line expressing a target protein with fluorescent molecules.

In this example, the first expression vector was pcDNA3.1+ expression vector in which the N-terminus of miniShank3 was linked to mEGFP as a fluorescence donor; the second expression vector was pcDNA3.1+ expression vector in which the N-terminus of miniShank3 was linked to mCherry as a fluorescence acceptor. 1.5 μg of two expression vectors were transfected into the COS-7 cell line using Lipo6000™ of Beyotime, and after the cells were cultured for 48 hours, data were collected by using a Plan-Apochromat 63x/1.4 Oil DIC M27 objective lens of Zeiss, with a pixel size of 40 nm×40 nm. The imaging for FRET efficiency was measured based on the sensitized emission method: the laser wavelengths for exciting the fluorescence donor and the fluorescence acceptor were 488 nm and 561 nm, respectively, and the following emission bands were collected: 490-560 nm (donor channel), 576-700 nm (FRET channel), and 576-700 nm (acceptor channel), respectively. The FRET efficiency was calculated based on formula 8 in the literature Gordon G. W. et al. (1998) Biophys J. 74 (5): 2702-13 and formula 2 in the literature Xia Z. and Liu Y. (2001) Biophys J. 81 (4): 2395-402.

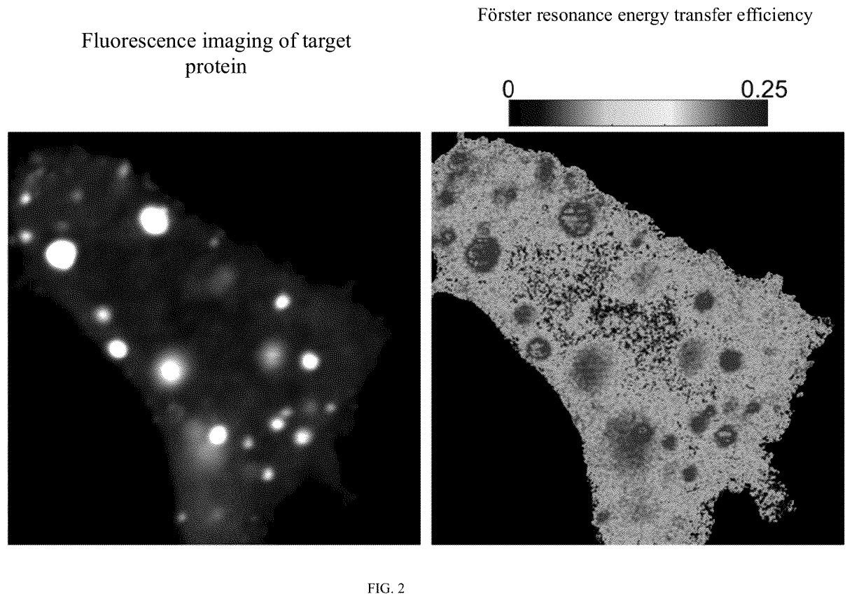

As shown in FIG. 2, when the synaptic protein miniShank3 was overexpressed in COS-7 cells, a liquid-liquid phase separation phenomenon occurred spontaneously (left graph), and there was a significant difference between the FRET efficiency inside the condensed phases (average 0.22) and that outside the condensed phases (average 0.11) (right graph), thus proving the principle of the present invention, and the phase transition process of the target protein was detected by measuring the change in FRET efficiency.

It is obvious that the above examples are merely illustrative for a clear explanation and are not intended to limit the embodiments. Various changes and modifications can be made by those of ordinary skills in the art on the basis of the above description. It is unnecessary and impossible to exhaustively list all the embodiments herein. Obvious changes or modifications derived therefrom still fall within the protection scope of the present invention.

What is claimed is:

1. A detection method for protein liquid-liquid phase separation (LLPS) and phase transition, comprising the following steps:
constructing a first expression vector for expressing an energy donor fluorescent molecule fused to a target protein and a second expression vector for expressing an energy acceptor fluorescent molecule fused to the target protein;
transferring the first and the second expression vector into primary cultured cells;
stimulating the primary cultured cells while collecting fluorescence microscopic imaging data by using an appropriate fluorescence microscope; and
calculating a change in Förster resonance energy transfer (FRET) efficiency as a proxy for monitoring the process of LLPS and phase transition of the target protein.

2. The detection method according to claim 1, characterized in that the target protein is a neuronal synaptic protein;
the primary cultured cells are primary cultured neurons.

3. The detection method according to claim 1, characterized in that the second expression vector comprises 2-3 energy acceptor fluorescent molecule nucleotide sequences in tandem.

4. The detection method according to claim 1, characterized in that the primary cultured cells transfected with the first and the second expression vectors therein are stimulated by an electric field produced by an electric field generator.

5. The detection method according to claim 4, characterized in that the electric field has a strength of 25-50 V/cm and a frequency of 20-50 Hz.

6. The detection method according to claim 1, characterized in that the fluorescence microscope is installed with two or more excitation lasers or emission filters.

7. The detection method according to claim 1, characterized in that the Förster resonance energy transfer efficiency is determined based on a sensitized emission method, a ratiometric method, or a fluorescence lifetime imaging method.

8. The detection method according to claim 1, characterized in that the Förster resonance energy transfer efficiency is determined based on the sensitized emission method.

9. The detection method according to claim 1, characterized in that the detection method is used for detecting a process of LLPS and phase transition of a protein in the synapse of living neurons.

10. Use of the detection method of claim 1 in drug target screening.
